(19) United States
(12) Patent Application Publication
(10) Pub. No.: US 2008/0194516 A1
(43) Pub. Date: Aug. 14, 2008

(54) SYSTEM FOR MANAGING ANTI-COAGULANT INFUSIONS IN PATIENTS

(76) Inventor: Samuel J. Flanders, Indianapolis, IN (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 11/934,297

(22) Filed: Nov. 2, 2007

Related U.S. Application Data
(63) Continuation-in-part of application No. 11/131,707, filed on May 18, 2005.

(51) Int. Cl. A61K 31/715; A61K 31/47; A61P 7/02

(52) U.S. Cl. 514/54; 514/314

(57) ABSTRACT

An anti-coagulant maintenance system for use in the treatment of patients with deep-vein thrombosis (DVT), pulmonary embolism (PE), Acute Coronary Syndrome, need for prophylactic anticoagulation or another related condition. The system establishes and adjusts the dosing of an anti-coagulant based upon periodic blood testing of a system specified frequency. Warning or alert messages or signals are produced if blood tests are not performed and a dosage adjustment is made based upon a coagulation test result outside of an established normal range. It is particularly useful for patients in a hospital or in-patient environment.

### CROSS REFERENCE TO RELATED APPLICATIONS

The present application is a continuation-in-part of U.S. patent application Ser. No. 11/131,707 filed May 18, 2005 entitled “System for Managing Glucose Levels in Patients with Diabetes or Hyperglycemia.”

### FIELD OF THE INVENTION

This invention relates to maintenance of anti-coagulant levels in patients with deep-vein thrombosis (DVT), pulmonary embolism (PE), Acute Coronary Syndrome, or another related condition, and in particular, to a system that aids in the correct continued administration of an anti-coagulant through the use of computerized dosage calculations that are made with the use of individual-specific information and test results. In addition, the invention may be used in the prevention of blood clots in high risk patients.

### BACKGROUND

Maintaining proper anti-coagulant levels, such as those indicated by an activated partial thromboplastin time (commonly known as PTT), is important for many patients, e.g., patients suffering from deep-vein thrombosis (DVT) or pulmonary embolism (PE), in order to prevent further complications. One exemplary anti-coagulant is heparin, which often has immediate but somewhat unpredictable results. Heparin combines with other factors in the blood to inhibit conversion of prothrombin to thrombin and fibrinogen to fibrin. Established clots are not dissolved but the heparin prevents further clot formation and allows the body to naturally dissolve existing clots. Heparin has an average half-life of 30-180 minutes. This half life can be prolonged by higher doses, liver disease or kidney disease. Due to this relatively short half-life, patients are frequently dosed by using a combination of bolus doses and continuous infusions.

The need to control the anti-coagulant levels is even more important with bridged warfain therapy patients in critical care situations, such as in hospital intensive care units following surgical procedures, as those patients are no longer able to continue their oral treatment and thus must be moved to an another delivery method, such as intravenous delivery. Compounding this problem is the fact that anti-coagulant levels in such patients may be unstable, necessitating frequent measurements and adjustments of administered anti-coagulant dosage. At times the degree of anti-coagulant dosage adjustment may be significant, or the calculated dosage amount may be high, so that it is difficult to determine whether the calculated anti-coagulant dose is correct or if an error in anti-coagulant levels measurement or an equipment malfunction has occurred.

### SUMMARY

It is therefore an object of one embodiment of the present invention to provide a system for monitoring anti-coagulant levels in a patient, calculating proper anti-coagulant dosages, and providing relevant feedback information and messages to the individual, or when used in a hospital or other in-patient setting, to the patient's physician, nurse, or other caregiver. Variations of activated partial thromboplastin time results outside a predetermined range results in more frequent measurements, while calculated anti-coagulant doses that fall outside normally expected levels (either high or low) generate feedback messages and warnings that require additional measurement or caregiver intervention to insure the correct treatment is administered.

### BRIEF DESCRIPTION OF THE DRAWINGS

FIG. 1 is a diagrammatic view of an anti-coagulant management system in accordance with one embodiment of the present invention.

FIG. 2 is a flowchart illustrating the operation of an anti-coagulant management system in accordance with an aspect of the present invention.

FIG. 3 is a diagrammatic view of an anti-coagulant management system in accordance with another embodiment of the present invention.

FIGS. 4 through 27 are representative computer screen images illustrating aspects of the operation of an anti-coagulant management system in accordance with an embodiment of the present invention.

### DETAILED DESCRIPTION

For the purposes of promoting understanding of the principles of the invention, reference will now be made to the embodiments illustrated in the drawings and specific language will be used to describe the same. It will nevertheless be understood that no limitation of the scope of the invention is hereby intended and alterations and modifications in the devices, systems and representations illustrated in the drawings, and further applications of the principles of the present invention as illustrated herein being contemplated as would normally occur to one skilled in the art to which the invention relates.

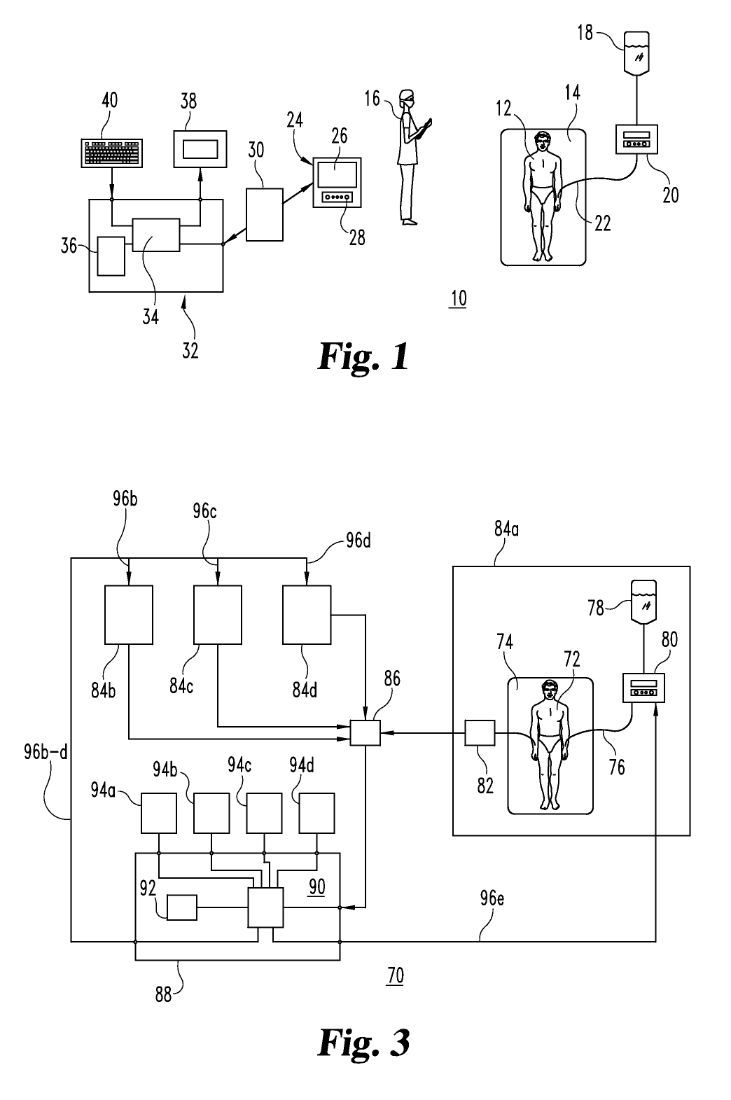

Referring to FIG. 1, there is shown an anti-coagulant management system 10 for a patient 12 who is illustratively being cared for in a hospital critical care setting, e.g., within an intensive care unit following surgery, although other patient settings are of course possible. The condition of patient 12 on bed 14 is shown as being illustratively monitored directly by a nurse or caregiver 16, but at least some functions that are performed by nurse 16 could be performed by automatic monitoring (pulse, blood pressure), data entry, and/or intravenous medication delivery equipment (not shown), to name only a few possible examples. For purposes of explaining an embodiment of the present invention, patient 12 is shown as receiving a continuous drip of an anti-coagulant from reservoir 18 that is controlled by drip regulator 20 through an intravenous (IV) line 22.

In operation, nurse 16 draws a blood sample from patient 12 by a known, available means including, but not limited to, a venipuncture using known, commercially available products. Using the drawn blood, a coagulant test, such as an activated partial thromboplastin time test (hereinafter a PTT test) or an activated clotting time test (hereinafter ACT test) is performed at the request of the nurse, by an automated machine, or by some other entity, such as a lab technician. For purposes of clarity, the illustrative embodiment will be described with reference to a PTT test as the coagulation test, however, it shall be appreciated by one of skill in the art that any coagulant test may be utilized.

Upon receiving the results of the PTT test, the nurse 16 enters the PTT results of the test performed on the blood drawn from patient 12 into data handling device 24. Device 24 is illustratively shown as having a display 26 and an input 28. Display 26 may be of any conventional or available display type, such as, for example, a CRT or LCD screen, while input 26 may be a computer keyboard, for example. When the patient's PTT test results have been entered into device 24, the entered information is sent via communications channel 30 to computer or data processor 32 which may be located at a central location, such as a nurses' station or hospital-wide patient monitor center. Communications channel 30 may be of the form of a hardwired connection, a local area network, a wireless network, or an internet-based wide area network, to cite a few non-limiting examples. Network access may advantageously provide access to patient data from other hospitals or in-patient facilities, and it can allow patent 12 to be moved within a networked facility or between network-linked facilities, while still maintaining active monitoring of the patient's condition and providing access to historical patient data.

Data processor 32 illustratively comprises a central processing unit (CPU) 34 and memory 36, which may be of any known or available form, such as, for example, ROM, PROM, RAM, EPROM or EEPROM. Also shown as being connected or associated with data processor 32 are display 38 (such as, for example, a CRT or LCD screen) and input device 40, such as a keyboard, for example.

Data processor 32 evaluates the PTT test results of patient 12 to determine if the current anti-coagulant dosage level is too high or too low. If it is too high, data processor 32 calculates an appropriate reduced dosage that is most likely to bring the PTT test results of patient 12 back within a predetermined range, such as specified by a nomogram. If the current anti-coagulant dosage rate is too low, data processor 32 calculates an increased dosage that is most likely to safely return the PTT test result of patient 12 to the desired range. If the PTT test result is within the desired range for patient 12, data processor 32 calculates a proper anti-coagulant dosage rate sufficient to maintain the PTT test result of patient 12 within the desired range. The information is sent back to device 24 via communications channel 30 where it appears on display 26. Nurse or caregiver 16 then makes any necessary adjustments to drip regulator 20 so that the proper amount of anti-coagulant from reservoir 18 is delivered to patient 12. The calculation used by CPU 34 of data processor 32 illustratively utilizes a known algorithm identified described in an article entitled “Heparin and Low-Molecular-Weight Heparin,” by Jack Hirsh, CM, MD, FCCP and Robert Raschke, MD, MS presented at The Seventh ACP Conference on Antithrombotic and Thrombolytic Therapy, but a proprietary algorithm or some other proven calculation could be developed or adapted to be suitable as well.

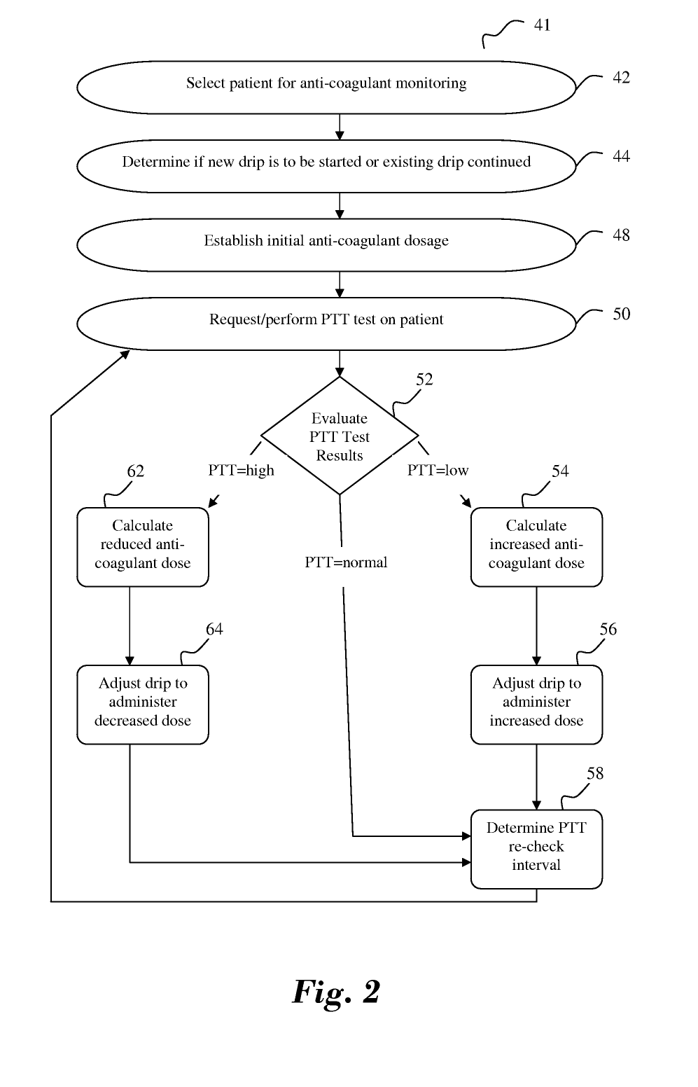

FIG. 2 illustrates a flowchart, with continued reference to FIG. 1, which shows an anti-coagulant management process 41 in accordance with an embodiment of the present invention, which will be used to illustrate the manner in which anti-coagulant management system 10 of FIG. 1 operates. Beginning at step 42 of process 41, a particular patient is selected for anti-coagulant monitoring or management by system 10. For illustrative purposes, the process of step 42 selects critical care patient 12. Step 44 determines from information provided by step 42 whether patient 12 requires an anti-coagulant drip to be established, or whether a previously established anti-coagulant drip is to be restarted. If the drip is to be restarted, information concerning the previous anti-coagulant drip for patient 12 is retrieved from database 36 and used in step 48 to set initial anti-coagulant administration, e.g., drip rate and desired PTT range, data for patient 12. Having access to known information specific to patient 12 greatly aids in establishing an initial anti-coagulant dosage/infusion rate that will be more accurate, and hence lead to a quicker stabilization of the PTT test results for patient 12 than would be possible if a drip rate were started from a nominal average value. Access to this information allows patient 12 to be temporarily discontinued from a drip without requiring a “break-in” period when the drip is resumed. If patient 12 is a new patient, or if information related to a previous drip is no longer valid, certain initial parameters, such as high and low target PTT results that establish a desired range for patient 12, are set and stored in database 36. Process 41 then proceeds to step 50, which performs a PTT test on a blood sample from patient 12. In FIG. 1, the PTT test result associated with patient 12 is measured manually by nurse 16, and entered into data handling device 24, which communicates that information to data processor 32. Process 41, including step 50, need not be performed manually by a nurse or caregiver, but could be performed automatically without human intervention. The measured PTT result is then evaluated at step 52 to determine if the current PTT level falls below, within, or above the desired range previously established for patient 12.

If the PTT test result of patient 12 is low (i.e., below the lower limit of the desired PTT result range), step 54 calculates the appropriate increase in the anti-coagulant infusion rate needed to bring the PTT results of patient 12 into the desired range. This information is used by process 41 at step 56 to administer the increased anti-coagulant dose to patient 12. Process 41 then sets an appropriate recheck interval at step 58, e.g., 360 minutes, at which time an alarm either provides an audible or visible alert to nurse 16 that it is time to perform another PTT test on patient 12. In an automated arrangement, the alarm could initiate a new PTT test via step 50 directly. The information determined at step 58 is also stored in database 36 to provide historical data that can be used to generate a complete report about patient 12, re-establish a drip, or more accurately predict the course of treatment needed to control anti-coagulant levels in patient 12. Once the interval expires the process repeats itself again beginning from step 50.

If the evaluation at step 52 determines that the PTT test result is high (i.e., a time above the upper limit of the desired PTT time range), process 41 calculates the proper reduced anti-coagulant drip rate for patient 12 at step 62. This anti-coagulant infusion is then administered at step 64, which permits adjustment of the anti-coagulant infusion rate and volume being given to patient 12 based on the information determined at step 62. At step 58, process 41 then determines an appropriate interval for performing the next PTT test (that is also stored in database 36) for patient 12. At the end of such interval, alarm 68 provides an alert to a nurse or caregiver that it is time to perform an additional PTT test on patient 12 or, in an automated system or environment, directly initiates a PTT measurement via step 50. Once the interval expires the process repeats itself again beginning from step 50.

Process 41 may incorporate additional alerts that require additional evaluations or determinations in order to proceed, so that improper measurements or data entry, or an equipment malfunction, may be discovered before an incorrect treatment is administered to a patient. Such alerts may, for example, be associated with step 50 if the PTT results measured at a given time is significantly different than that measured previously, or if the previous measurement was done only a short time before. Alerts could also be associated with steps 54 or 62 if the calculated anti-coagulant infusion rate appears to be abnormally high or low. Other alert mechanisms may also be included as desired and the actions needed, e.g., doctor or nurse sign-off, PTT test recheck, may be specified in order for the process to proceed. Process 41 can use the information stored in database 36, which creates a permanent archived record for each patient, to create individual patient reports or to determine trends and predictions from statistically analyzing a large amount of data from a number of patients.

Many of the functions of system 10 that have been described with reference to FIG. 2 may be performed by electronic circuitry and/or with computer software, including but not limited to the steps of determining whether measured or calculated amounts are within normal ranges, issuing alert messages, and setting patient PTT test intervals.

FIG. 3 shows an anti-coagulant management system 70 that is adapted to handle multiple or even large numbers of patients,, and that automatically performs certain functions that were done manually or with human intervention in system 10 of FIG. 1. It is understood, however, that the number of patients and the degree of automation are shown and described for illustrative purposes only, and the aspects and advantages of the present invention are not dependent upon any particular number of patients or the manner in which the operating steps are performed.

System 70 operates similarly to system 10 of FIG. 1 in that an illustrative patient 72, located on bed or other device 74 in a hospital or other in-patient facility, is administered an anti-coagulant drip via IV tube 76 from reservoir 78, with the rate and amount of that drip being set by drip controller 80. The PTT result of patient 72, however, is measured automatically by measurement device 82, rather than manually as was done as by nurse 16 in system 10 shown in FIG. 1. For the sake of a more simplified explanation of system 70, the above-described elements associated with patient 72 can be considered to make up an overall patient unit 84a.

FIG. 3 illustratively shows additional patient units 84b, 84c, and 84d, although it is understood that any number of patient units may be provided. The automatically measured PTT levels from each of patient units 84a-d is applied to controller 86 which coordinates data from the various patient units. The data is provided to data processor 88 in a manner that enables data processor 88 to identify the patient unit that is the source of each piece of information. It is understood that the functionality of controller 86 may be incorporated in some other component such that an identifiable discrete element may not be present. Data processor 88 illustratively incorporates a central processing unit (CPU) 90 and a memory unit 92, which may be of any known or otherwise appropriate type. Also shown as being associated with data processor 88 are input/output (I/O) devices 94a-d, which illustratively correspond to patient units 84a-d respectively. It is of course possible that a single or limited number of I/O devices may be provided so that multiple patient units can be accommodated by one I/O device in a multiplexed fashion. Anti-coagulant drip rate information is provided to the corresponding drip controllers in each of patient units 84a-d via output lines 96a-d.

The operation of system 70 in FIG. 3 may also be represented by the process that is shown and described in connection with FIG. 2. Alert signals may be evaluated by automatic failsafe or double checking circuitry, or a human intervener, such as the equivalent of nurse 16, may be called upon to verify the accuracy of measurements or calculations if they do not appear to fall within “normal” or previously measured limits.

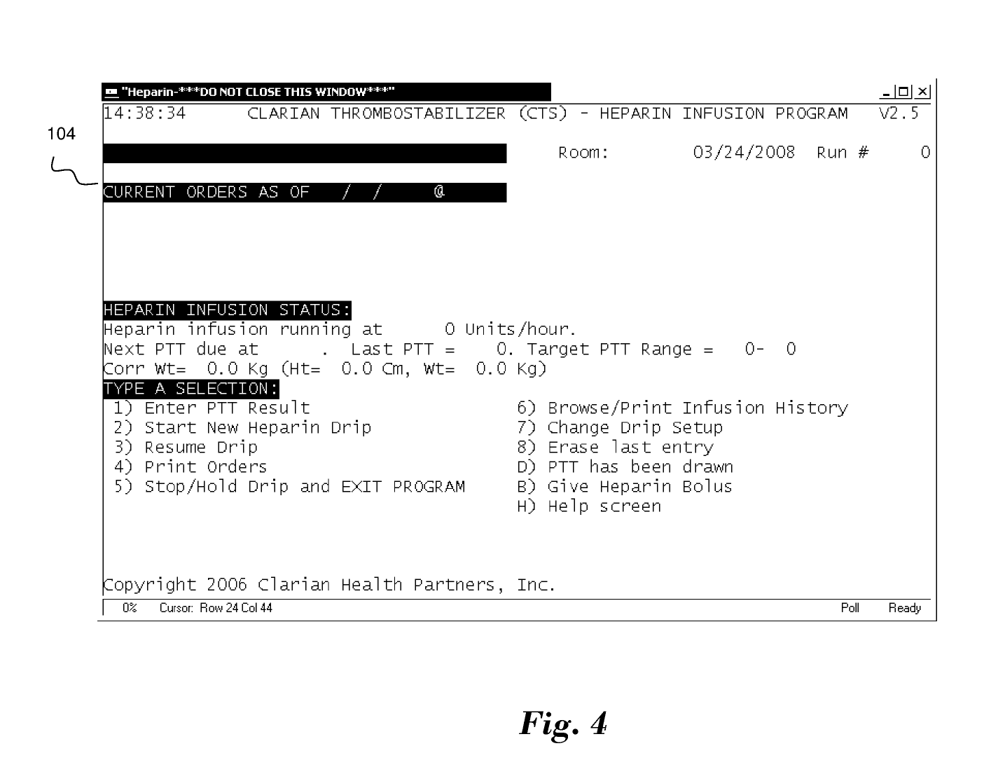

FIGS. 4 through 25 will now be described as illustrative examples of representative display screens for a computer software program that performs certain of the functions described in connection with system 10 and/or system 70. For purposes of clarity, the display screens are illustrated as suitable for use with heparin, a common and widely used anti-coagulant. However, it shall be appreciated that other anti-coagulants may be utilized within the system. For the sake of efficiency and readability in describing FIGS. 4 through 26, reference will be made solely to system 10, although it is understood that references to the operation of system 10 are also applicable to system 70 whenever such described function is performed by system 70.

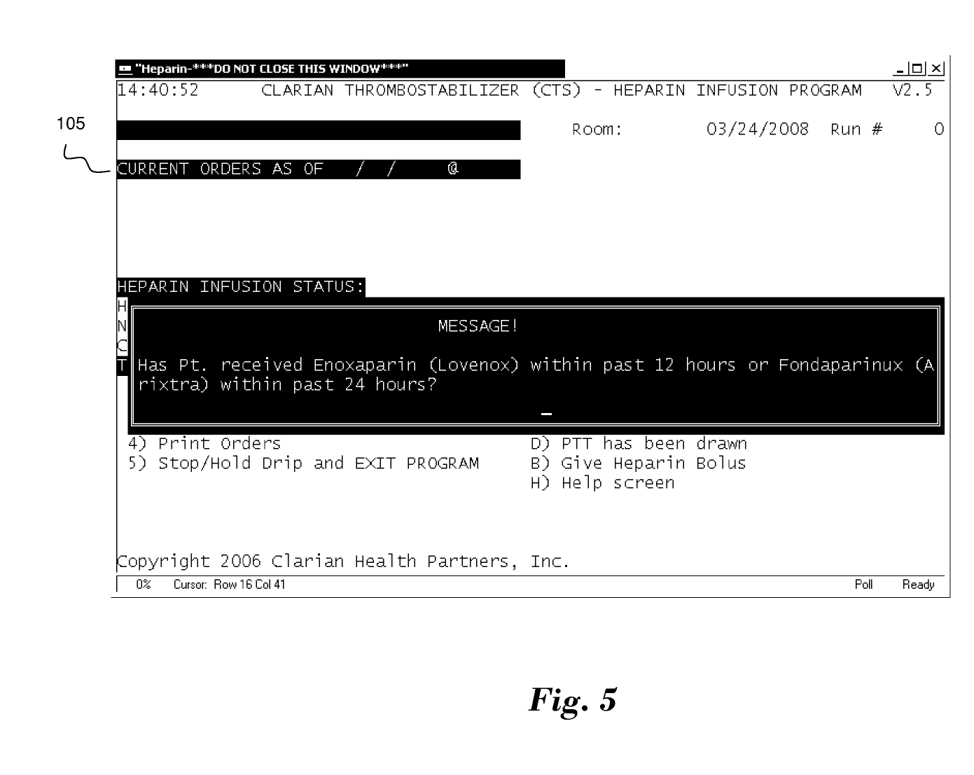

FIG. 4 illustrates one example of an initial system screen 104 suitable for use in anti-coagulant management system 10. Screen 104 includes fields for a patient name or other identifier as well as other relevant data. It is understood that the information provided on screen 104 is shown for illustrative purposes, and other types of information, such as the ages of the patients, their physical description, and a medication list, for example, could also be shown. Screen 104 further provides a list of possible actions that may be taken with respect to a given patient, including the action of entering a PTT result, starting a new heparin drip, resuming a previous, e.g., temporarily discontinued, drip, or placing a hold on a currently active drip. If the action to resume a prior drip is selected, for example, a screen (not shown) might then be displayed to verify that the action of resuming a prior drip was intended to be selected to improve accuracy and prevent errors. FIG. 5 shows a display screen 105 that displays an alert message in response to a selection of function 1) Start New Heparin Drip. The alert message prominently displayed on the screen requires a response as to whether the patient has taken a drug which may cause a potentially harmful reaction in a recent time period. It shall be appreciated that other warning indications may be presented in response to a new heparin drip request to enhance patient safety.

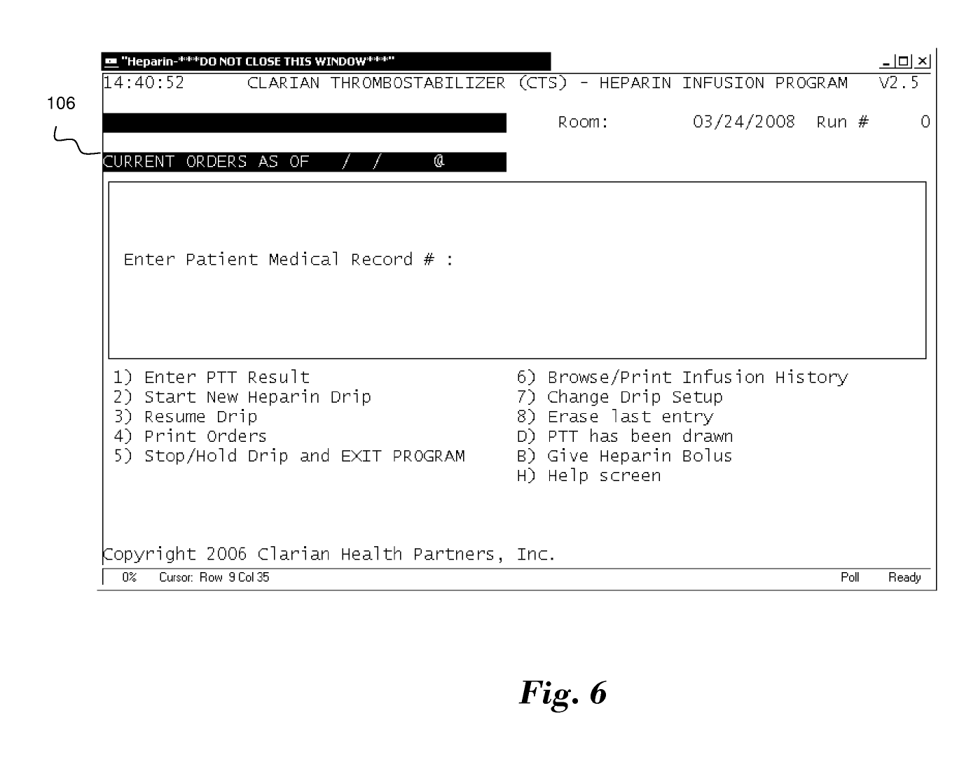

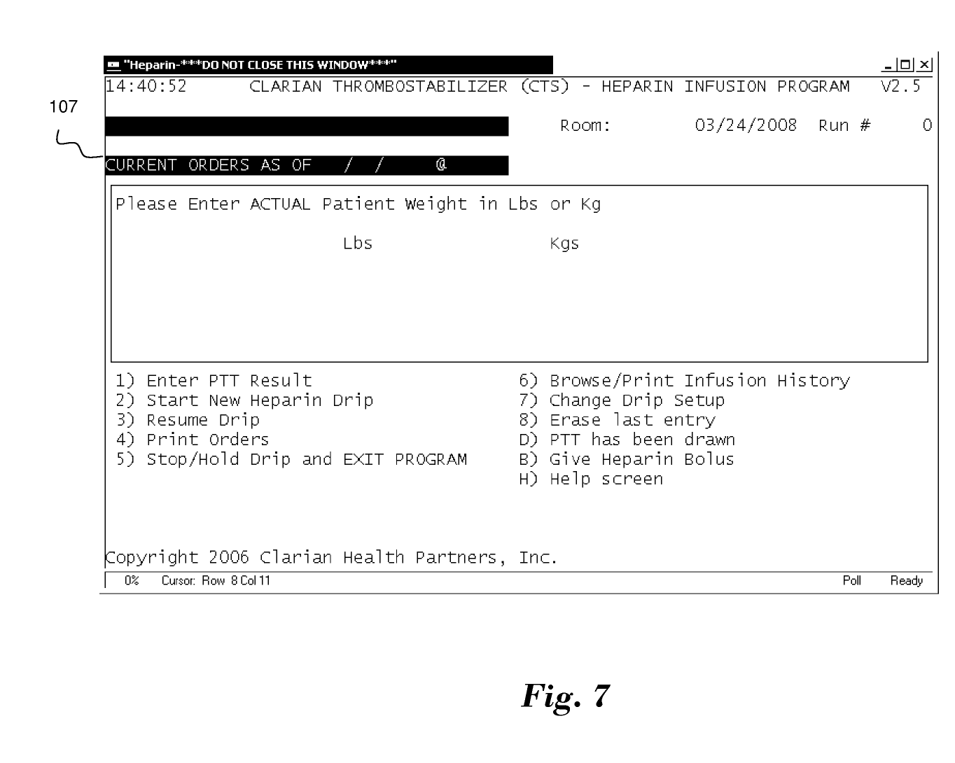

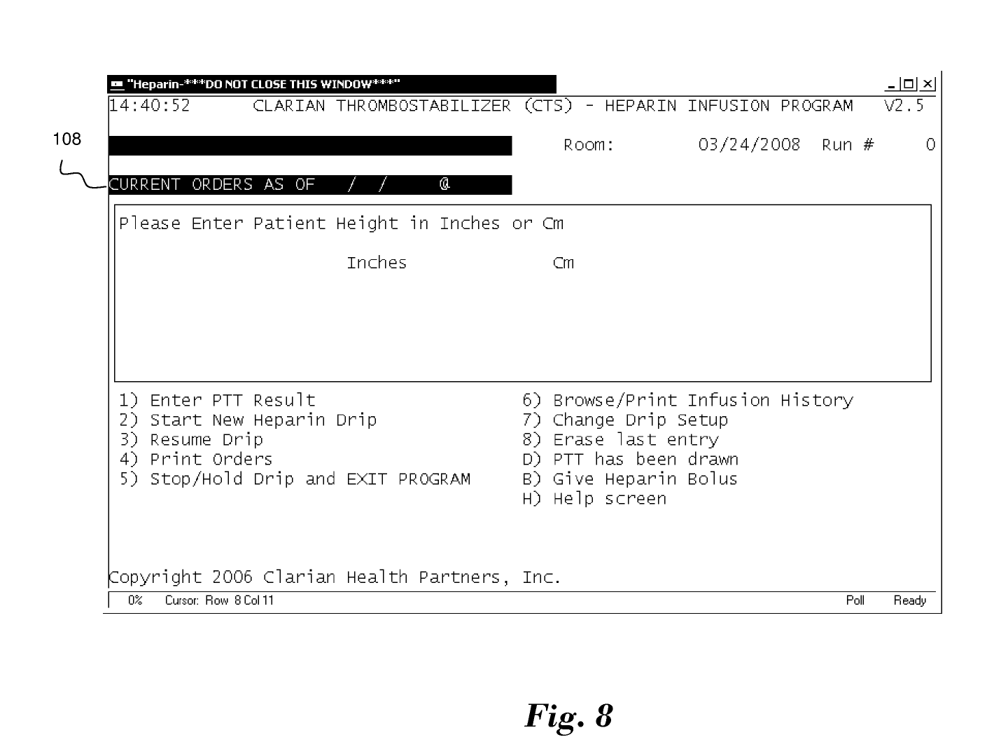

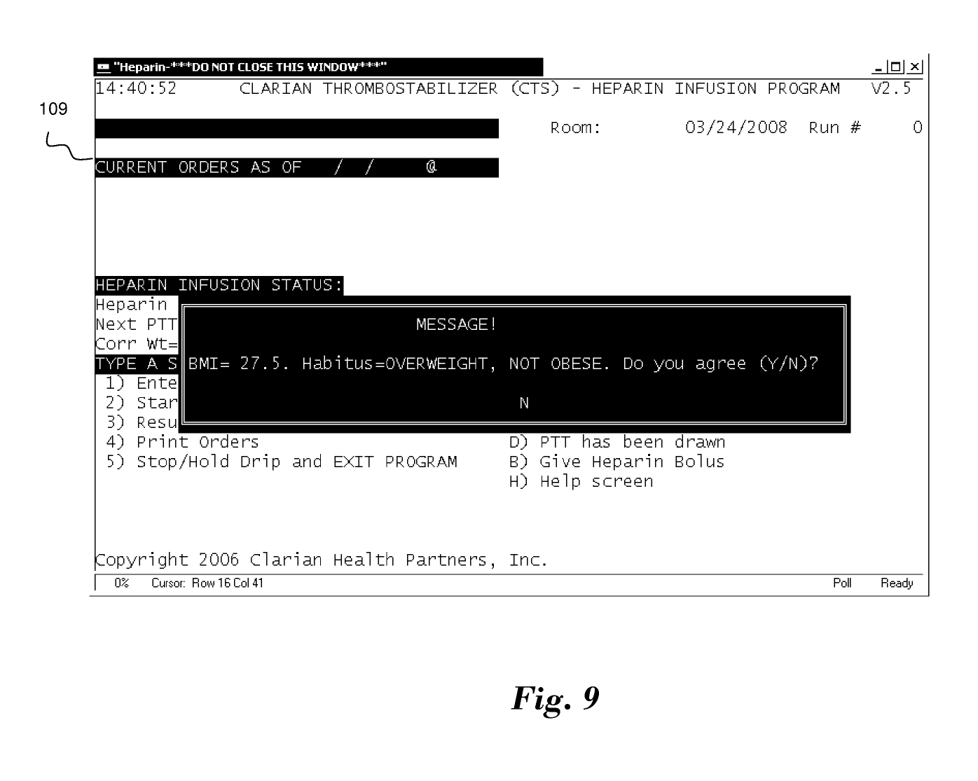

FIG. 6 illustrates a screen 106 that illustratively appears in response to a decision to establish a new heparin drip for a patient, i.e., Mickey Mouse. In the illustrated embodiment, the system requests the Patient Medical Record # of the patient. In alternate embodiments the system may request any other identifier as utilized by the hosting facility. FIGS. 7 through 20 illustrate a series of screens that requests specific information that system 10 will use to determine heparin drip rates for the current patient Mickey Mouse. The information provided for Mickey Mouse would therefore be expected to be different than corresponding information for other patients. By way of example, FIG. 7 shows a screen 107 that requires the entry of the selected patient's (Mickey Mouse's) weight, either in pounds or kilograms, while FIG. 8 shows a screen 108 requesting an entry of the selected patient's height, in inches or centimeters. FIG. 9 shows a screen 109 which presents an assessed body mass index (BMI) of the current patient based upon the information entered in the prior screens. As such, the attending nurse or other user is able to quickly confirm the accuracy of the entered data by confirming the assessment of the system. It shall be appreciated that the system 10 may retrieve height, weight, and any other type of information associated with a patient identifier from a central database if desired.

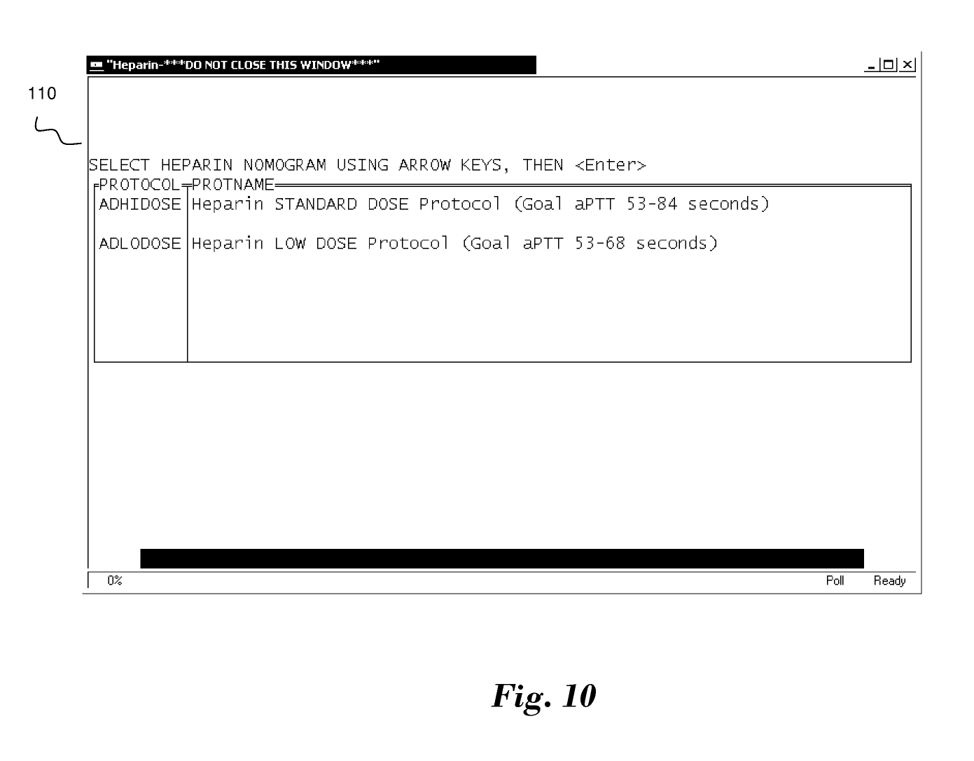

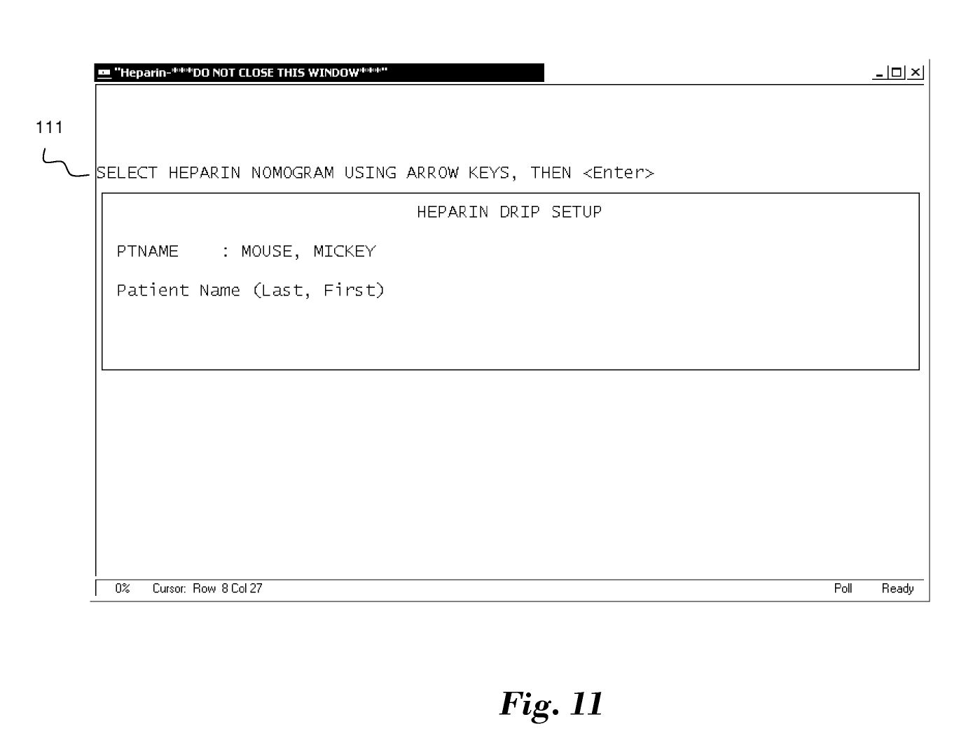

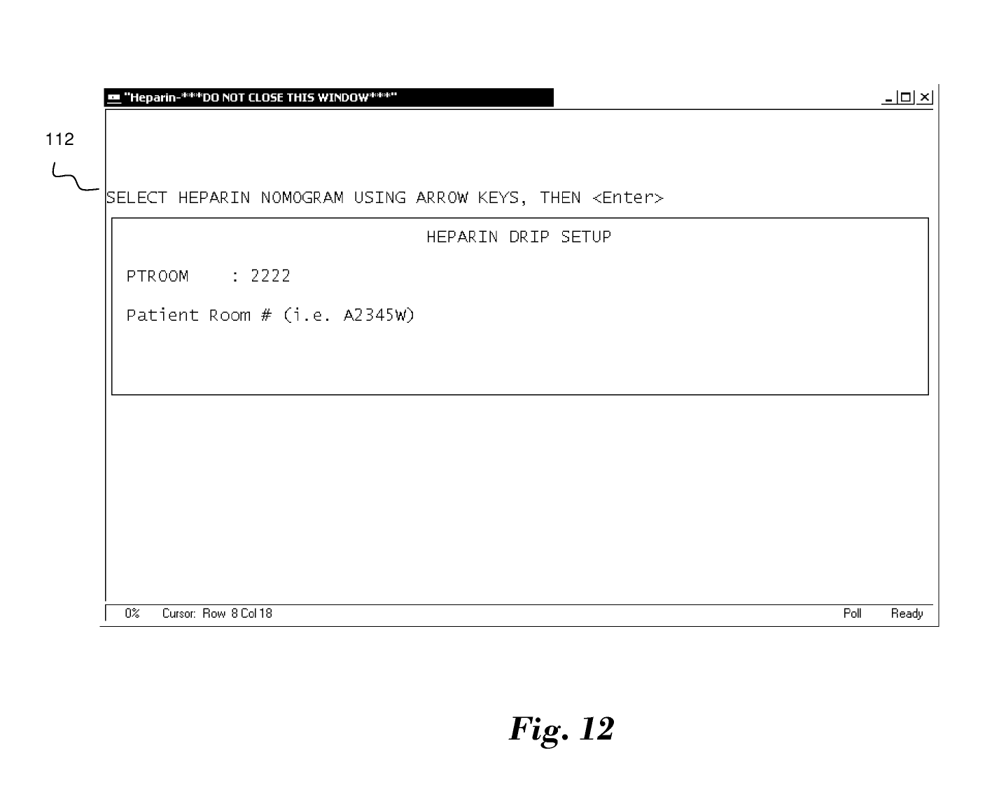

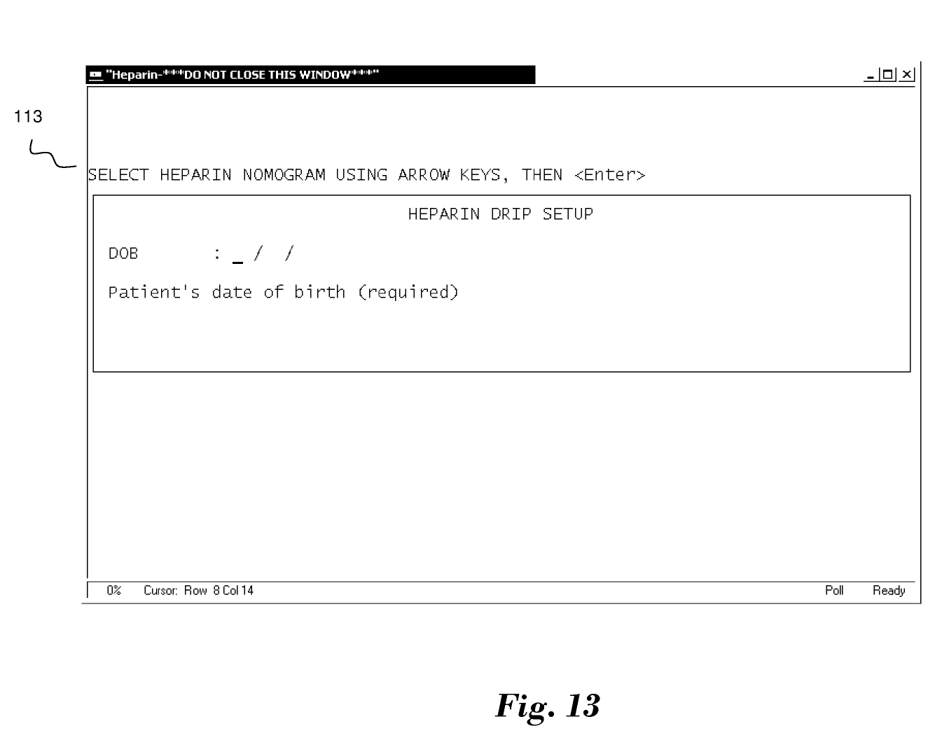

FIG. 10 illustrates a screen 110 in which the heparin dosage is selected by selecting from a set of predetermined heparin nomograms. In the illustrated embodiment, a high dose and low dose nomogram for an adult is provided. FIGS. 11-13 request additional patient information that may be supplied by the user or alternatively obtained from a connected database in association with the patient identifier provided. FIG. 11 shows a screen 111 for entry of the patient's name, FIG. 12 shows a screen 112 for receiving the current room of the patient, and FIG. 13 shows a screen 113 for entry of the patient's date of birth.

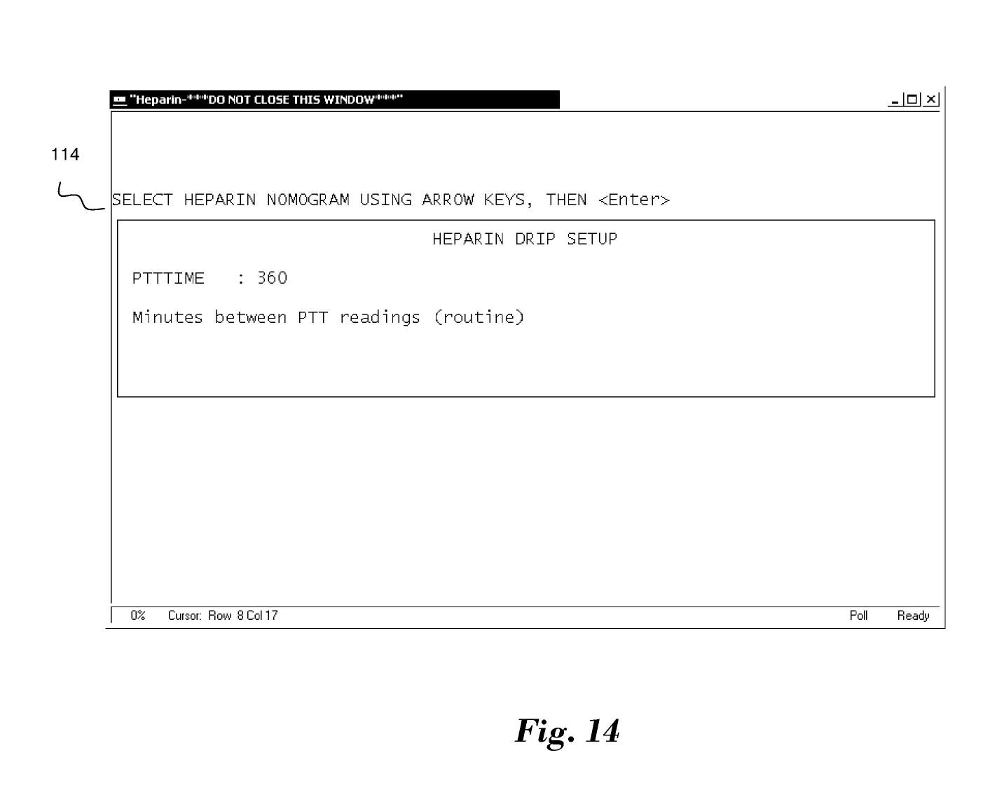

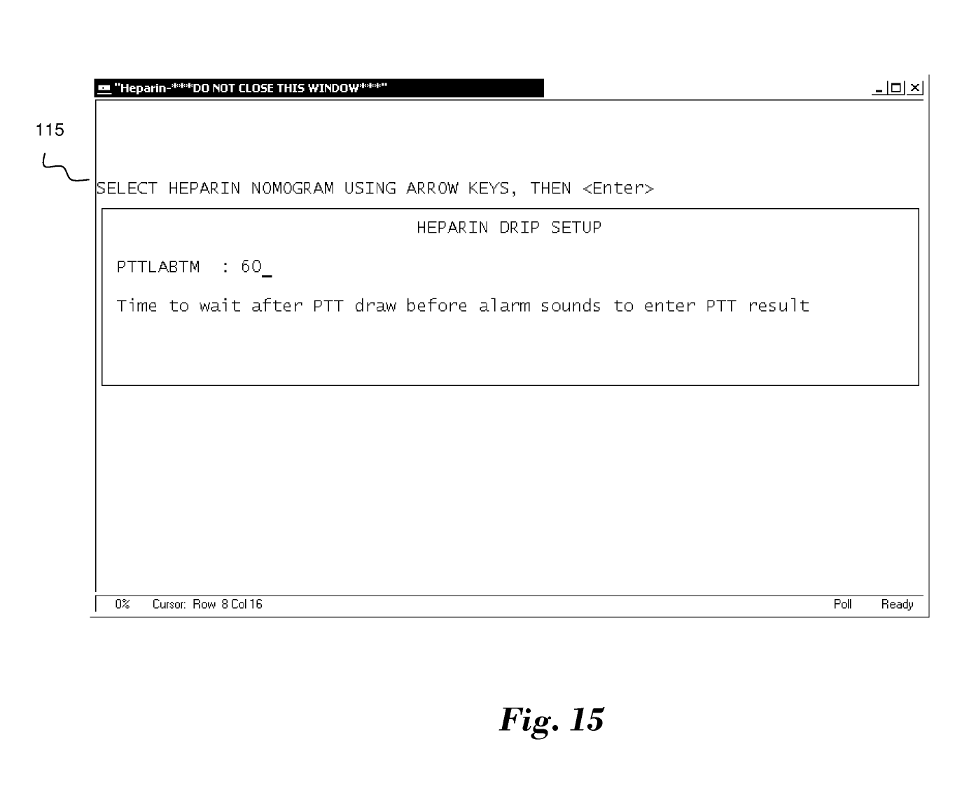

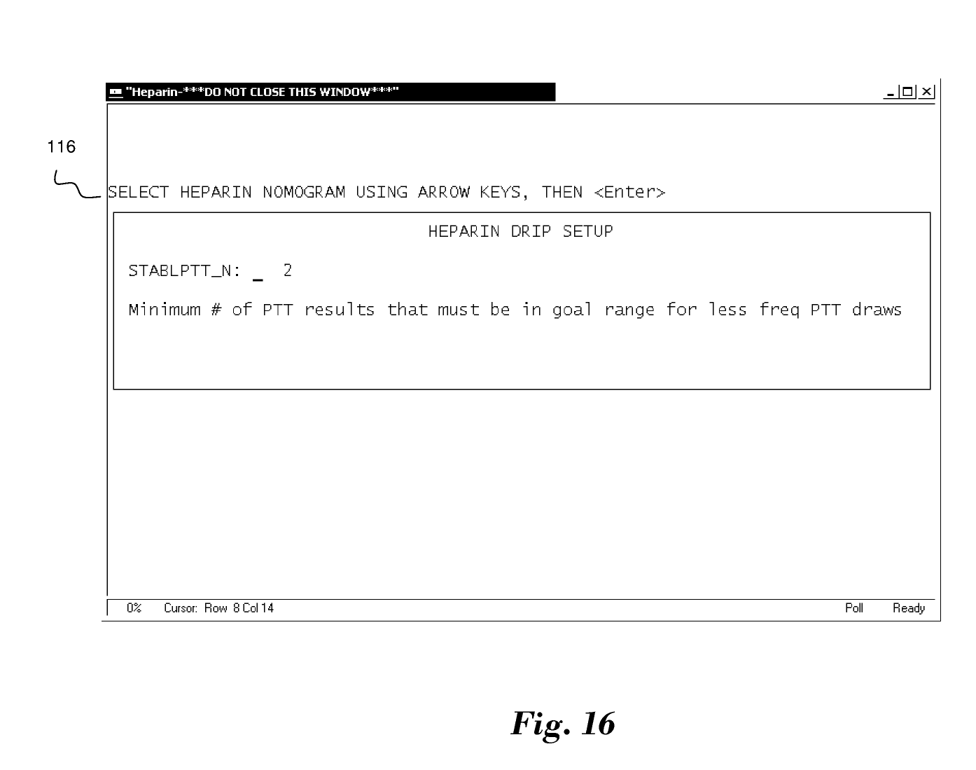

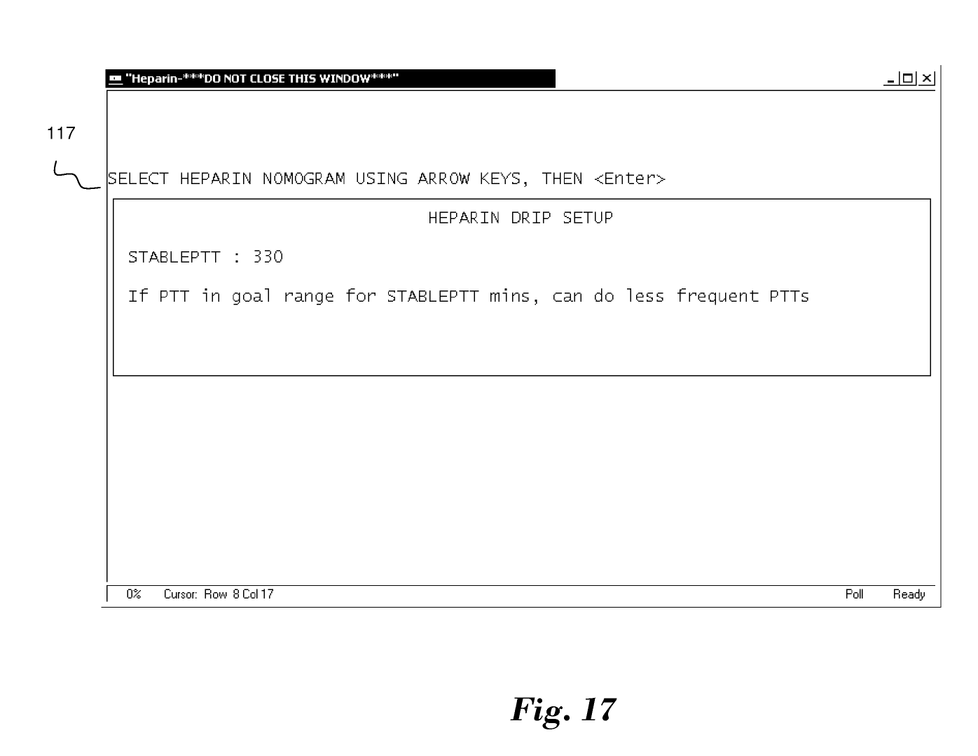

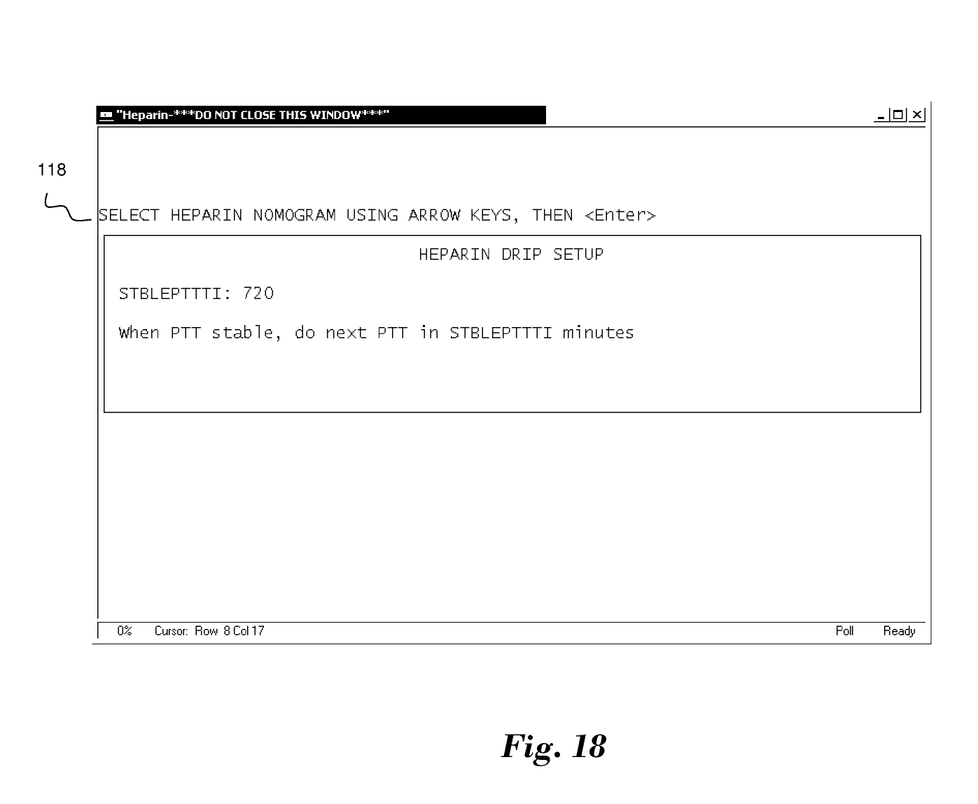

FIG. 14 illustrates a data entry section on screen 114 that specifies the number of minutes system 10 should wait between PTT test requests. The default range is 360 minutes (6 hours). FIG. 15 then shows in screen 150 a request for the number of minutes that system 10 should wait for the user to confirm performance and enter the results of a PTT test after the time period entered in screen 114 has elapsed prior to sending an alarm. FIG. 16 shows a screen 116 in which a data entry is requested to specify how many consecutive PTT test results must be within the desired range prior to reducing the frequency of PTT tests. Additionally, FIG. 17 shows screen 117 which also requests a minimum number of minutes that the PTT test results must be in the desired range for the system 10 to lower the frequency of PTT tests. Then, screen 118 of FIG. 18 shows a request for the number of minutes that the time between PTT test requests may be increased to upon stable PTT test results in conformance with the data entered in the prior two screens.

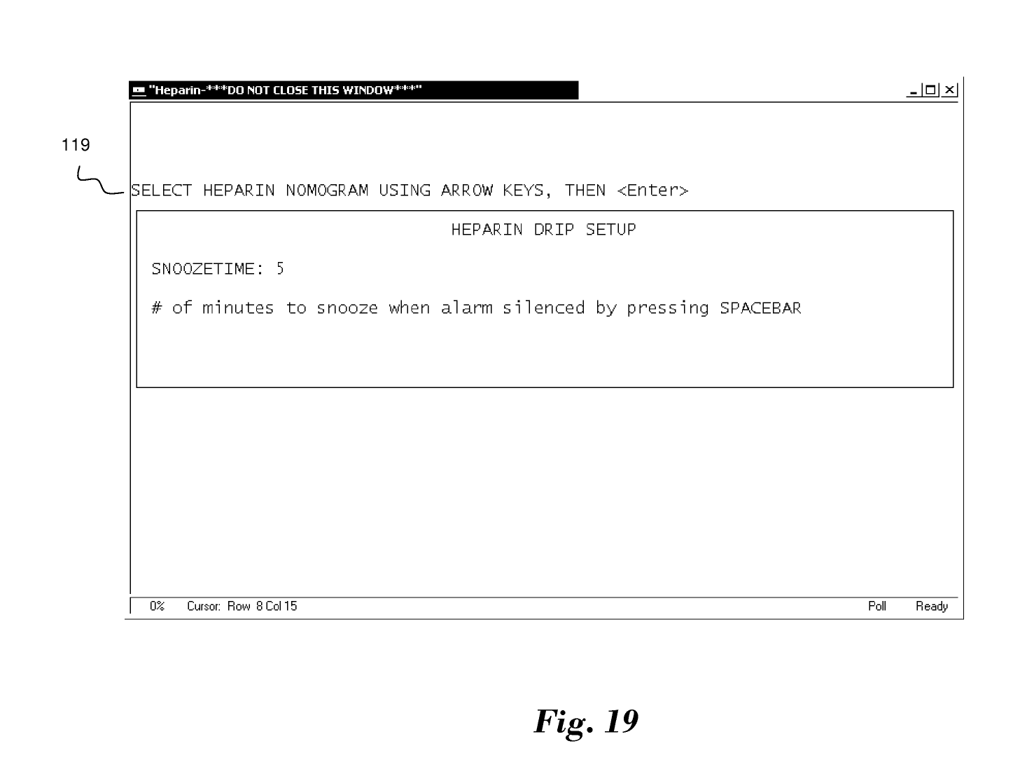

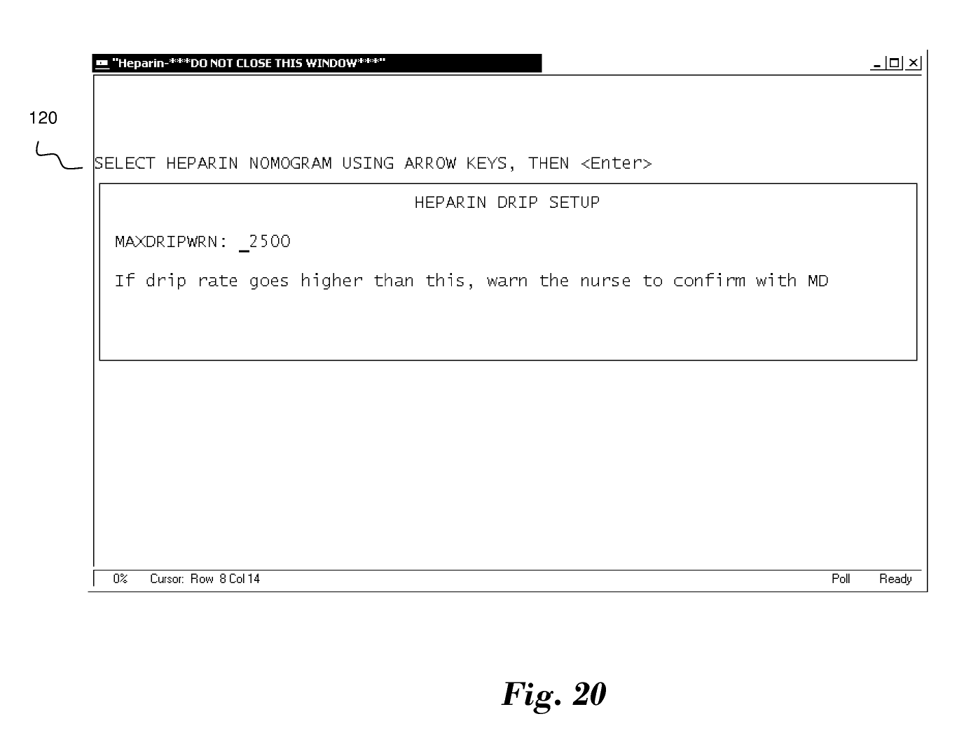

As additional configuration options, screen 119 of FIG. 19 allows for the entry of a snooze time when an alarm indicating the need for a PTT test is silenced and, FIG. 20 shows a screen 120 which allows for the entry of a maximum drip rate allowed before an independent check or confirmation, such as by the patient's doctor, is needed to proceed. The snooze time is an interval that allows a nurse or caregiver to silence an audible alert or alarm for a limited time if it is not possible to immediately address the underlying condition that caused the alert. It is of course possible that in an automated system, such as system 70, for example, the alert is generated via a software program that causes the program to determine the proper treatment and automatically administer the necessary drugs or solutions to the patient.

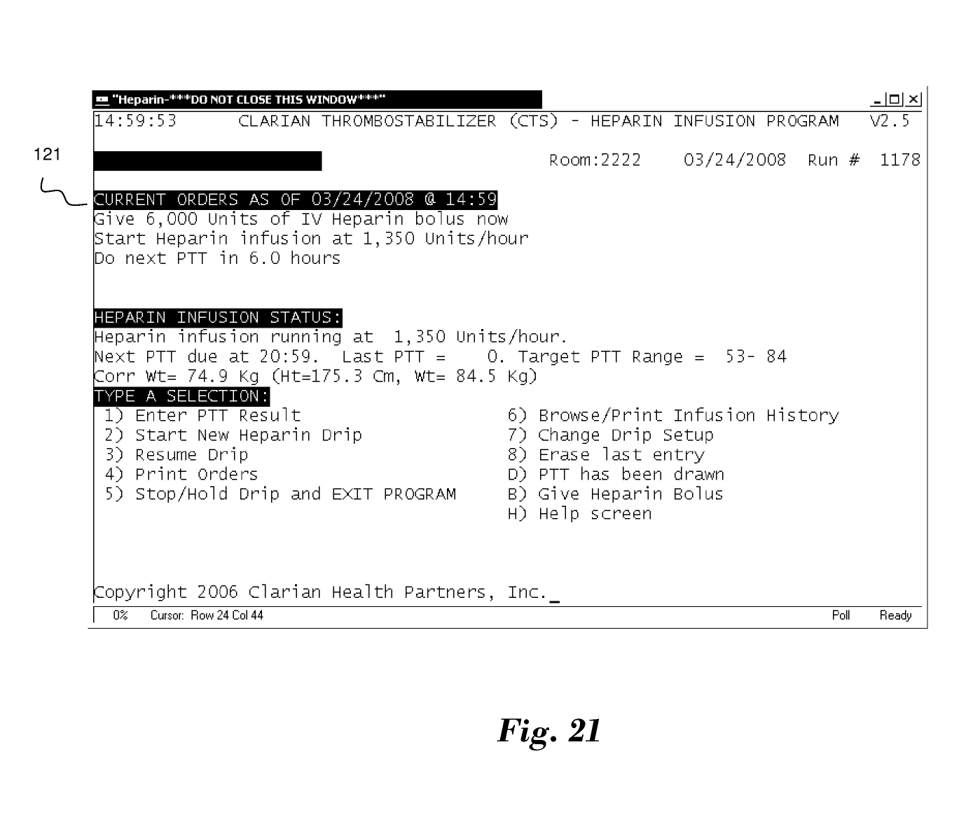

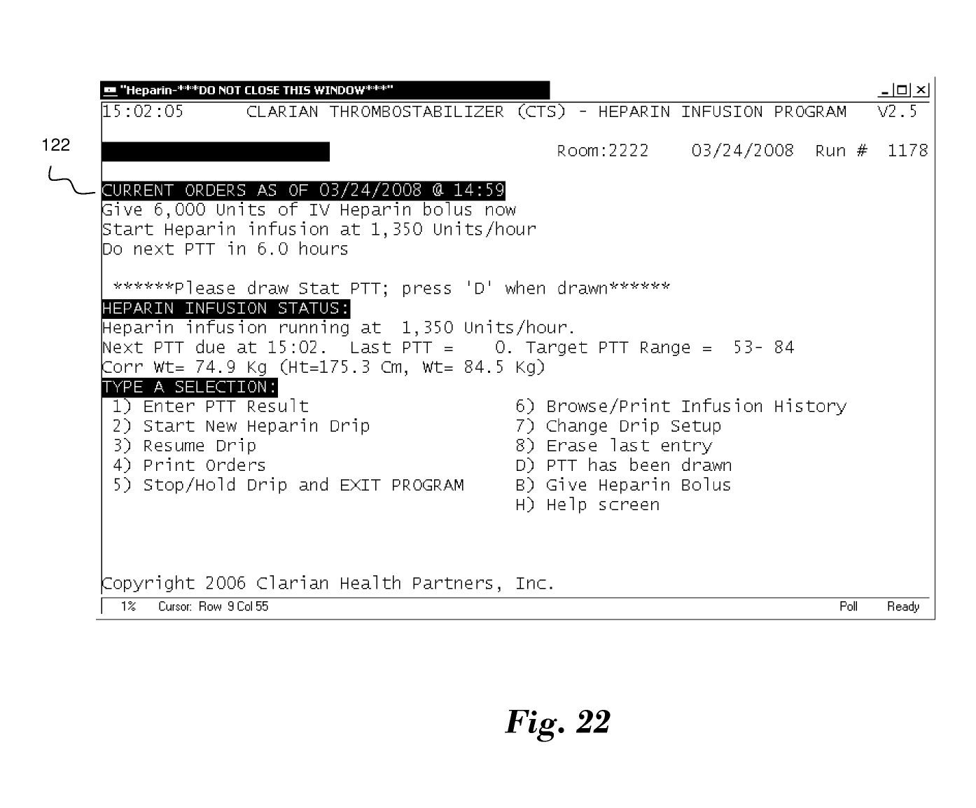

Once configured, FIG. 21 shows a screen 121 suitable for use by system 10 indicating orders derived for a heparin dose as specified by the patient information and selected nomogram. In this example, the screen 121 included orders to provide a 6,000 unit bolus dose of heparin in conjunction with a 1,380 Units/hour heparin drip. The orders, with the calculated heparin dosage, may be caused to be displayed at the patient's bedside and/or at a central monitoring station, and be of a visual and/or auditory nature, so that nurse 16 is appropriately notified to modify the heparin infusion rate to address the patient's PTT results. The alert may also cause the necessary amount of heparin to be administered directly to the patient without any human intervention. The next PTT test is required in 6 hours. Upon expiration of this time, the screen display of system 10 changes to one such as screen 122 of FIG. 22. Screen 122 displays an alert, which may be combined with an auditory or other indication, to request that a PTT test be performed.

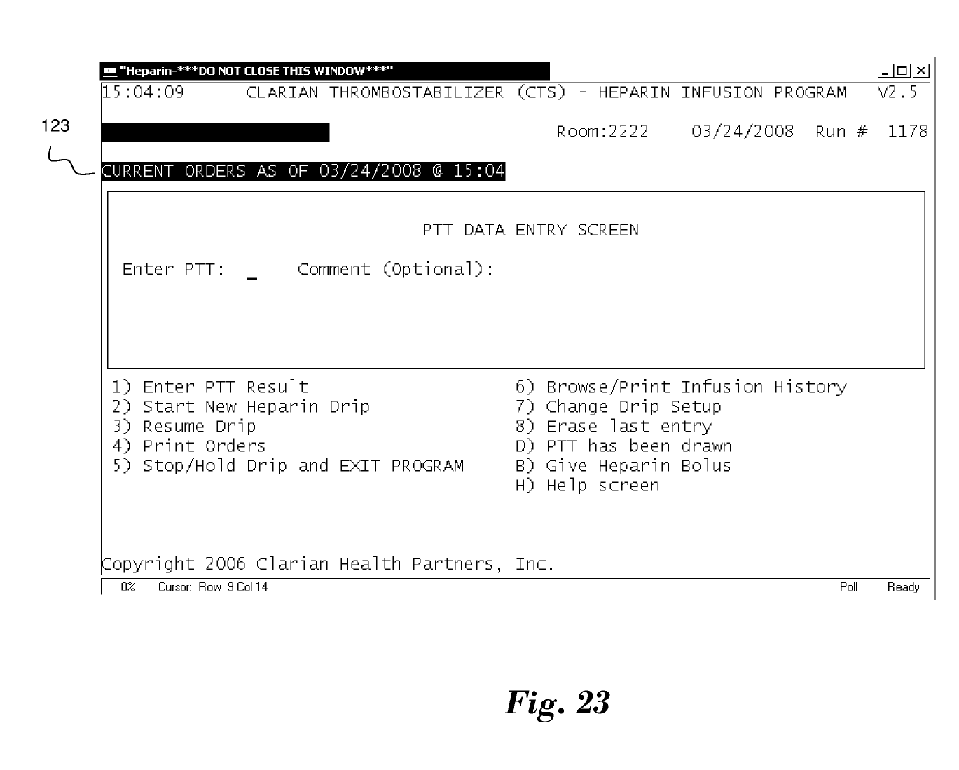

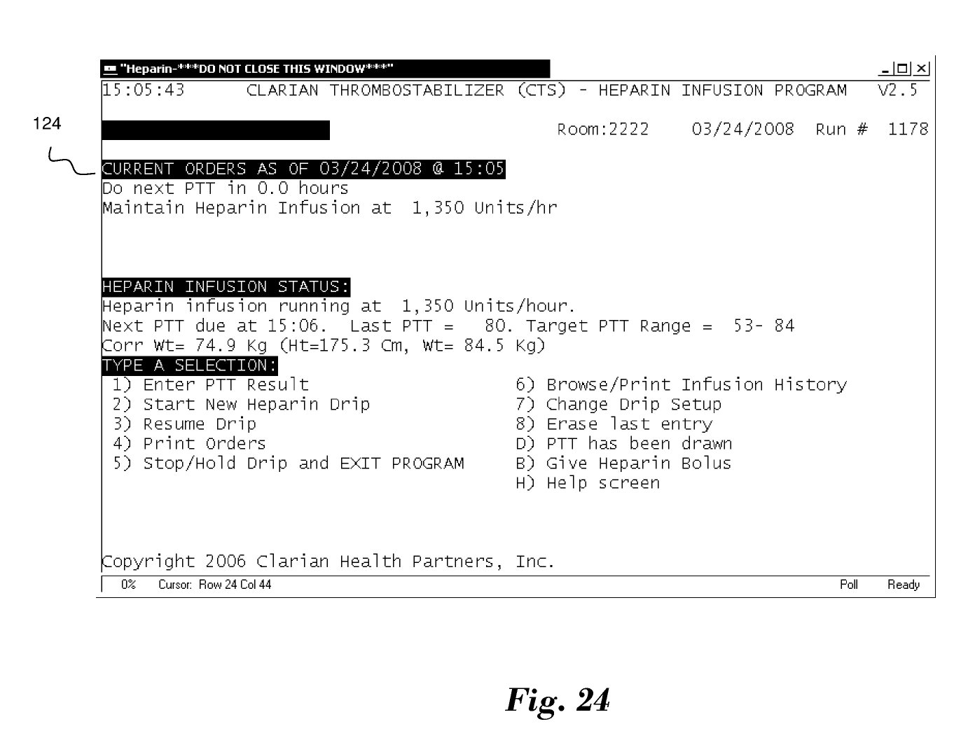

Once performed, the user may interact with system 10 through screen 123 of FIG. 23 to enter the results of the PTT test. Based upon these results, the system 10 will calculate new orders and display them on screen 124 of FIG. 24. In this example, the orders request the heparin drip to be maintained at its current rate in light of PTT results of 90, which are well within the predetermined range of 61-104.

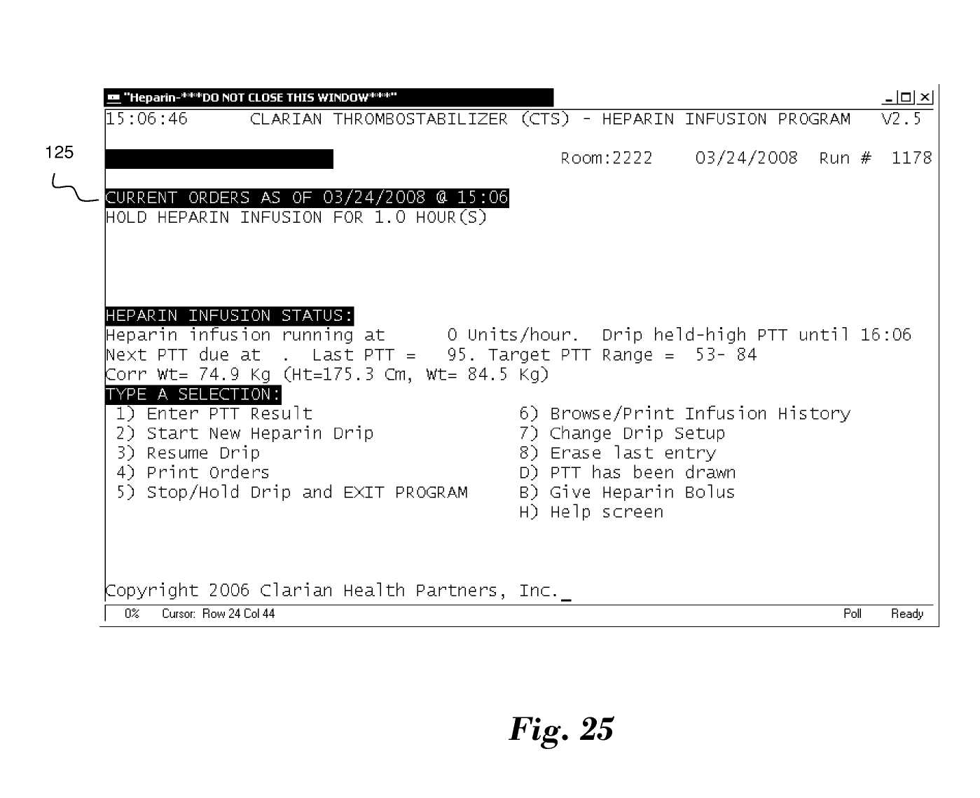

Alternatively, in light of PTT test results of 120, as shown by screen 125 of FIG. 25, the system 10 orders the user to hold the heparin infusion for one hour (0 units/hour) in order to allow the heparin levels to decrease to conform PTT results within the desired range.

The previous description has been made based on treatment of patients in an in-patient medical/surgical setting, such as a hospital or nursing home, as the novel features of the invention lend themselves particularly well to a critical or intensive care setting. The scope of the invention, however, is not limited to an in-patient environment. Significant advantages can also be realized by ambulatory or otherwise healthy individuals with a need for anti-coagulants through the use of, for example, periodic or continuous heparin infusions. The manner in which such as system, incorporating one or more embodiments of the present invention, could provide automatic PTT tests and administration of proper heparin infusion amounts while still maintaining sufficient safeguards to protect against an inadvertent application of an incorrect dose due to an equipment malfunction or some incident of human error.

While the invention has been illustrated and described in detail in the drawing and foregoing description, the same is to be considered as illustrative and not restrictive in character, it being understood that only the preferred embodiment has been shown and described and that all changes, modifications and equivalents that come within the spirit of the inventions disclosed are desired to be protected. The articles “a”, “an”, “said” and “the” are not limited to a singular element, and include one or more such elements.

What is claimed is:

1. A system for maintaining an anti-coagulant level in an individual comprising:
setup means for establishing a desired range of results for a blood coagulation assay of the individual;
measuring means for determining a blood coagulation assay result for the individual;
calculating means responsive to said blood coagulation assay result for determining a proper anti-coagulant dosage amount for the individual when said blood coagulation assay result is above or below said desired range;
delivery means for administering said anti-coagulant dosage amount to the individual;
evaluation means responsive to said anti-coagulant level for determining a time interval for said individual after which said blood coagulation assay result of the individual is to be re-measured; and
timing means for generating a signal when said time interval has elapsed.

2. The system of claim 1, wherein said measuring means operates on a periodic basis.

3. The system of claim 1, wherein said measuring means operates on a continuous basis.

4. The system of claim 1, wherein said calculating means is further responsive to at least one criteria associated with the individual.

5. The system of claim 1, wherein said criteria comprises previously measured blood coagulation assay results associated with the individual.

6. The system of claim 1, wherein said criteria comprises previously administered anti-coagulant dosages for said individual.

7. The system of claim 1, wherein said criteria comprises information regarding a previous bolus anti-coagulant dose.

8. The system of claim 1, wherein said anti-coagulant is heparin.

9. The system of claim 1, wherein said anti-coagulant is Argatroban.

10. The system of claim 1, wherein said individual is a patient in a hospital.

11. The system of claim 1, wherein said proper anti-coagulant dosage is a function of a nomogram.

12. The system of claim 1, wherein said blood coagulation assay is an activated partial thromboplastin time (PTT) test.

13. The system of claim 1, wherein said blood coagulation assay is an activated clotting time (ACT) test.

14. The system of claim 1, further comprising estimating means responsive to said blood coagulation assay result or said anti-coagulant dosage amount, and to at least one criteria associated with said individual, for estimating the accuracy of said calculated dosage amount.

15. The system of claim 14, further comprising notification means for generating an alert signal when said estimated accuracy is below a predetermined level.

16. The system of claim 15, wherein said alert signal is auditory.

17. The system of claim 14, wherein said alert signal is visual.

18. The system of claim 1, wherein said timing means signal is applied to said measurement means for causing said measurement means to measure a blood coagulant assay result for said individual.

19. The system of claim 1, wherein said delivery means comprises an intravenous infusion pump.

20. A system for maintaining anti-coagulant levels in a plurality of individuals comprising:
setup means for establishing a desired range of blood coagulation assay test results for each one of said individuals of said plurality of individuals;
measuring means for determining a blood coagulation assay result for each one of the individuals;
calculating means responsive to said blood coagulation assay result for each one of the individuals for determining a proper anti-coagulant dosage amount for each one of said individuals whose blood coagulation assay result is below or above said desired range for the individual;
delivery means for administering said proper anti-coagulant dosage amount to each one of the individuals;
evaluation means responsive to said blood coagulation assay result of each one of the individuals for determining a time interval for each one of the individuals after which said blood coagulation assay test result is to be re-measured; and
timing means for generating a signal when said time interval for each of one of the individuals has elapsed; and
data storage means for maintaining information from at least one of said setup means, said measuring means, said calculating means, said delivery means, said evaluation means, or said timing means for each one of the individuals.

21. The system of claim 20, wherein said calculating means is further responsive to at least one criteria associated with each one of the individuals.

22. The system of claim 20, further comprising reporting means coupled to said data storage means for generating time-based reports for each one of the individuals.

23. The system of claim 20, further comprising reporting means coupled to said data storage means for generating dosage-based reports for the plurality of individuals.

24. A method for maintaining anti-coagulant level in an individual comprising the steps of:
establishing a desired range of blood coagulation assay results for the individual;
determining a blood coagulation assay result for the individual;
calculating in response to said assay result a proper anti-coagulant dosage amount for the individual when said assay result is outside said desired range;
administering said anti-coagulant dosage amount to the individual;
determining in response to said assay result a time interval for the individual after which said individual's assay result is to be re-measured; and
generating a signal when said time interval has elapsed.

25. A method for maintaining anti-coagulant level in a plurality of individuals comprising the steps of:
establishing a desired range of anti-coagulant levels for each one of the individuals of the plurality of individuals;
measuring a PTT test result for each one of the individuals;
calculating in response to said PTT test result for each one of the individuals a proper anti-coagulant dosage increase amount for one each of the individuals whose PTT test result is below said desired range for the individual and for calculating an anti-coagulant dosage decrease amount for each one of the individuals whose PTT test result is above said desired range for the individual;
administering at least one of said anti-coagulant dosage increase amount or said proper anti-coagulant dosage decrease amount to each one of the individuals;
determining in response to said PTT test result for each one of the individuals a time interval for each one of the individuals after which the individual's PTT test result is re-measured;
generating a signal when said time interval for each one of the individuals has elapsed; and
maintaining information derived from at least one of said steps of establishing, measuring, calculating, administering, determining, or generating.
